(19) United States
(12) Patent Application Publication
(10) Pub. No.: US 2005/0118156 A1
(43) Pub. Date: Jun. 2, 2005

(54) Storage-stable fibrin sealant

(76) Inventor: Christopher J. Woolverton, Kent, OH (US)

Correspondence Address: FLESHNER & KIM, LLP, P.O. BOX 221200, CHANTILLY, VA, 20153, US

(21) Appl. No.: 10/503,765

(22) PCT Filed: Dec. 4, 2002
(86) PCT No.: PCT/US02/38739

Related U.S. Application Data
(60) Provisional application No. 60/336,636, filed on Dec. 4, 2001.

(51) Int. Cl. A61K 38/46

(52) U.S. Cl. 424/94.6

(57) ABSTRACT

Provided are supplemented and unsupplemented, ready-to-use and instantly-available fibrin sealants (“FS”), prepared from ready-to-use, storage-stable, concentrated liquid fibrinogen preparations. The thus-produced FS product when applied to a tissue provides the elasticity, tensile strength, and adhesiveness necessary to prevent blood loss, to promote wound healing, and for many other therapeutic and non-therapeutic applications. Further provided are kits for, and methods of preparation of, the supplemented and unsupplemented, storage-stable FS products of the present invention, and methods of use and delivery therefor.

### BACKGROUND OF THE INVENTION

1. Field of the Invention

This invention relates generally to supplemented and unsupplemented, storage-stable fibrin sealants. More specifically, the subject invention relates to instantly-available fibrin sealants prepared from storage-stable, ready-to-use concentrated fibrinogen preparations, to methods of preparing such fibrin sealants, and to methods of use therefor to prevent blood loss, to promote wound healing, and for many other therapeutic and non-therapeutic applications.

Clot formation in humans, i.e., blood coagulation, occurs by means of a complex cascade of events in which in the final steps the monomeric form of fibrinogen reacts with thrombin and activated Factor XIII in the presence of calcium ions, to form a fibrin clot comprising a cross-linked fibrin polymer. Recently, biological adhesives have been developed comprising fibrinogen, thrombin and one or more other components, which imitate the final stages of natural coagulation, thereby resulting in a fibrin clot. Thus, the fibrinogen-based material, known as fibrin- or tissue-sealant, biological sealant, fibrin- or tissue-glue, biological adhesive, surgical adhesive, or the like (collectively referred to herein as a “fibrin sealant,” or “FS”), can be used to join living tissue together, and keep it joined to seal internal and external wounds, e.g., in tissue, organs, muscle, bones and skin, and to reduce blood loss while producing a hemostatic action (see for example patent FR-2448900). Such adhesives are commonly used in surgery, particularly to prevent or stop bleeding, replace or reinforce suture threads, hold grafts in place, e.g., skin grafts, to seal resectioned tissues, e.g., in lung or gastrointestinal tract surgery, or to glue parts of prostheses, etc.

FS products generally are prepared from: (1) fibrinogen concentrate, which may also contain fibronectin, Factor XIII, von Willebrand factor and traces of albumin; (2) an activator component such as thrombin (e.g., human or bovine) or a thrombin-like material; and (3) a thrombin activator, such as calcium ions (e.g., CaCl₂). The precise composition of each ES, however, is a function of the particular plasma fraction(s) used as the starting material. For example, commercially prepared FS products often contain bovine components. In Canada, Europe, and possibly elsewhere, commercially available FS typically also contains aprotinin as a stabilizer. Nevertheless, a direct relationship has been shown between tensile strength and the final fibrinogen concentration (Japanese Patent Unexamined Published Application, Kokai No. Sho 61-293443). Thus, the availability of concentrated fibrinogen is an important factor for the preparation of conventional FS products.

Australian Patent 75097/87 describes a one-component adhesive, which contains an aqueous solution of fibrinogen, Factor XIII, a thrombin inhibitor, such as antithrombin III, prothrombin factors, calcium ions, and, if necessary, a plasmin inhibitor. U.S. Pat. Nos. 4,427,650 and 4,427,651 (Stroetmann), describe the preparation of an enriched plasma derivative in the form of a powder or sprayable preparation for enhanced wound closure and healing that contains fibrinogen, thrombin and/or prothrombin, and a fibrinolysis inhibitor, and may also contain other ingredients, such as a platelet extract. U.S. Pat. Nos. 4,627,879 and 4,928,603 (Rose et al.), disclose methods for preparing cryoprecipitated suspensions that contain fibrinogen and Factor XIII and their use to prepare a FS. JP 1-99565 discloses a kit for the preparation of fibrin adhesives for wound healing.

PCT document WO91/09641 describes a fibrin glue containing fibrinogen and added thrombin. This FS contains added thrombin, but is prepared in such a way that the thrombin activity is inhibited, and in one embodiment it comprises no calcium ions, as these are not added until the time of use. However, the disclosed FS tends to coagulate spontaneously after about 90 seconds, even without the addition of calcium ions. When calcium ions are added, it coagulates in less than 2 seconds. In other embodiments, coagulation of the glue is slowed down by acidifying the product to a pH of less than 5.5 to inhibit thrombin activity. The disclosure further provides a means of increasing the pH at the time of use to nullify the inhibition effect,

In addition, FS delivery systems have been disclosed by Miller et al., U.S. Pat. No. 4,932,942 and Morse et al., PCT publication WO 91/09641. FS products have been commercially marketed for a number of years in Europe by Immuno AG (Vienna, Austria) and Behringwerke AG (Germany) (Gibble et al., Transfusion 30:741-747 (1990)) and elsewhere (see, e.g., U.S. Pat. Nos. 4,377,572 and 4,298,598, owned by Immuno AG).

However, most FS products used clinically outside of the U.S. pose certain risks and, as a result have not been approved by the Food and Drug Administration for use in the U.S.A. For example, as noted above, the FS products available in Europe contain proteins of non-human origin, e.g., aprotinin and bovine thrombin. Consequently, certain individuals are at risk of developing allergic reactions to such non-human protein additives. U.S. Pat. No. 6,183,498 reports that the use of biomedical adhesives have been observed to induce inflammatory tissue reactions.

Moreover, when heat inactivation is used to inactivate any viruses that may be present in the FS, the process may result in the formation of denatured proteins, which may also be allergenic. For example, the European heat inactivation methods do not inactivate prions which cause bovine spongiform encephalopathy (“mad cow disease”), which has been epidemic recently in bovine herds in European, and. hence disease could be carried in the bovine proteins used in the foreign FS products, risking human infection when those products are used for their intended purpose.

Alterbaum (U.S. Pat. No. 4,714,457) and Morse et al. (U.S. Pat. No. 5,030,215) disclose methods to produce autologous FS in which no bovine products are used. PCT publication WO94/07548 describes FS enriched with platelet factors that is able to coagulate without addition of thrombin by adding to the recalcified glue, a coagulation activator, such as kaolin. However, because the activator is not incorporated until the time the glue is used, the time coagulation time is uncertain and difficult to predict or control. This is because the fibrinogen concentrate is a highly viscous product, which is difficult to handle. Moreover, since coagulation progresses simultaneously with activation, it is difficult to separate the activator from time activated glue.

Nevertheless, at a sufficiently high fibrinogen concentration, FS preparations provide safe hemostasis, good adherence of the seal to the wound and/or tissue areas, high strength of the adhesions and/or wound sealings, and complete resorbability of the adhesive in the course of the wound healing process (Byrne et al., Br. J. Surg. 78:841-843 (1991)). For optimal adhesion, a concentration of fibrinogen of about 15 to 60 mg/ml is required in a ready-to-use tissue adhesive solution (MacPhee, personal communication). The clinical uses of FS products have been reviewed (e.g., by Brennan, Blood Reviews 5:240-244(1991); Gibble et al., Transfusion 30:741-747 (1990); Matras, J. Oral Maxillofac. Surg. 43:605-611 (1985); Lerner et al, J. Surg. Res. 48:165-181 (1990)).

Baxter/Hyland (Los Angeles, Calif.) in conjunction with The American National Red Cross have co-developed Tisseel, the first commercial fibrin sealant to be approved in the United States (see, e.g., U.S. Pat. Nos. 6,054,122; 6,117,425; and 6,197,325 (MacPhee et al.)). This FS product has advantages over those available in Europe because it is free of bovine proteins. For example, it contains human thrombin, and it contains no aprotinin, thereby reducing the potential for allergenicity. In addition, it is virally inactivated by a solvent detergent method, which produces fewer allergenic denatured proteins.

From the standpoint of preparation, the fibrinogen component of the FS can be prepared from plasma by cryoprecipitation, followed by fractionation, to yield a composition that forms a fibrin sealant, or clot, upon exposure to, or mixing with activated thrombin. In the prior art, the fibrinogen and thrombin concentrates are stored in lyophilized form that must be reconstituted and mixed with a solution of CaCl₂ immediately prior to use. Upon mixing, the components are applied to a tissue where they coagulate on the tissue surface and form a cross-linked fibrin clot, Factor XIII, which is present in the fibrinogen concentrate, catalyzes the cross-linking.

According to U.S. Pat. No. 5,290,552, early surgical adhesive formulations necessarily contained a high fibrinogen content (about 8-10%), from which lyophilates were extremely difficult to prepare. In fact, cryoprecipitates of concentrated fibrinogen are known to be highly unstable in liquid solution, thus requiring storage below −20° C. until use (http:www.tissuesealing.com/us/products/biological/monograph.cfm); i.e., in aqueous form concentrated fibrinogen is subject to spontaneous coagulation. Consequently, commercially available lyophilized and/or deep-frozen fibrinogen concentrates, such as Tissucol, must be liquefied, i.e., slowly thawed (“melted”) or reconstituted from lyophilates before application. Both liquefaction processes, however, are associated with significant effort and a considerable time lag before time product can be used in FS products, which can place an already injured patient into a life-threatening situation.

Therefore, significant effort has been undertaken to improve the solubility of lyophilized fibrinogen preparations. For example, one manufacturer requires the use of a magnetic stirrer added to the vials of protein to provide significant agitation while heating. This results in dissolution times which are faster than those obtained for the same product without significant mixing, but it still requires 30-60 minutes of preparation time simply to get the fibrinogen ready to use.

U.S. Pat. No. 5,962,405 provides storage-stable lyophilized or deep frozen liquid preparations of fibrinogen, which can be reconstituted and liquefied into ready-to-use fibrinogen and/or tissue adhesive solutions—preferably without the use of additional means, such as heating and/or stirring devices, to produce ready-to-use tissue adhesive solutions having a fibrinogen concentration of at least 70 mg/ml. The preparations comprise fibrinogen and at least one additional substance which improves the solubility of the preparations, and/or lowers its liquefaction temperature, and reduces the viscosity of a ready-to-use tissue adhesive solution at room temperature. However, because the liquefaction temperature is lowered, the '405 patent claims that liquefaction of the deep-frozen, concentrated fibrinogen solution is advantageously possible in a surrounding temperature of 20° to 23° C. (room temperature), as opposed to the previously required 37° C. warming conditions. Nevertheless, the method still requires storage under deep-frozen conditions (temperatures maintained at −15° C. to below −25° C.), and the preparations still take up to 15 minutes to liquefy.

Instructions for the previously mentioned Tisseel fibrin sealant (Baxter) indicate that preparation of the fibrinogen and thrombin components takes at least 15 minutes. The Baxter sealer protein concentrate (fibrinogen) is provided as a. freeze-dried powder, which is reconstituted by mixing with a fibrinolysis inhibitor solution. The Baxter thrombin component, which also comes as a freeze-dried concentrate, is reconstituted using a calcium chloride solution. Preparation of each component in the Baxter kit can be semi-automated using the optionally provided fibrinotherm heating and stirring device. To accommodate the protein preparation processes, each sealer protein concentrate vial contains a magnetic stir bar that fits into a custom-sized stirring well for uniform mixing at optimal physiologic temperature (37° C.).

However, not only does the need to slowly liquefy the protein components cause a significant delay in the formation of the FS preparation, a significant problem arises once fibrinogen is solubilized because its instability results in a tendency to prematurely self-coagulate. In fact, once prepared, the Baxter instructions indicate that the reconstituted solutions can be kept in their respective vials or syringes for a maximum of only 4 hours, after which any unused sealant must be discarded. As a result, the Baxter FS cannot be stored in a ready-to-use condition for any useful length of time.

As one solution to overcome the need to reconstitute or liquefy the lyophilized or deep-frozen fibrinogen products before use, especially concentrated preparations, certain fibrinogen preparations have been introduced which are soluble at room temperature. Unfortunately, however, such prior art products have proven to be cytotoxic (Beriplast, Biocol, Bolheal HG-4).

In an alternative solution, to delay the tendency of fibrinogen in aqueous solution to prematurely coagulate, U.S. Pat. No. 5,985,315 provides a stable biological pre-activated adhesive comprising fibrinogen with the addition of at least one activated coagulation factor whose activation does not depend on calcium ions. The preactivated adhesive is stable in aqueous solution, i.e., the solution does not coagulate spontaneously for at least one hour at a temperature of 20° C.; but it can be made to coagulate in about 5 minutes simply by adding calcium ions. No additional activator is required. Thus, the resulting biological adhesive requires neither the addition of thrombin or prothrombin to achieve coagulation. Unfortunately, however, 5 minutes is a very slow coagulation time, making the use of the resulting fibrin sealant impractical for use on any type of a flowing or pulsating wound, e.g., anastomoses, blood vessels, airholes in lung injuries, or injuries to parenchymal or bronchiole tissue.

From a medical standpoint therefore, the quick availability of ready-to-use, biological, tissue adhesives is essential, especially in surgical emergency situations. Despite continued advances in trauma care, a significant percentage of the population, both military and civilian, suffer fatal or severe hemorrhage every year. An alarming number of fatalities are preventable since they occur in the presence of those who could have achieved lifesaving control of their wounds, given adequate tools and training. Thus, there is a recognized need for an advanced, easy-to-use, field-ready hemostatic preparation, permitting not only trained medical personnel, but even untrained individuals to rapidly reduce bleeding in trauma victims. The effect of this need is two-fold: a significant number of trauma deaths could be prevented, and the demand upon the available blood supply could be reduced.

When presented with a large number of victims from severe natural or man-made disasters, local hospitals and clinics may be overwhelmed by the number of individuals requiring trauma care. Often the resulting demand for blood and blood products exceeds the locally available supplies; and in many cases, the demand for assistance exceeds the availability of trained medical personnel. However, the availability of a ready-to-use, self-contained FS preparation would permit local medical personnel and disaster relief workers to provide the injured with temporary treatment until definitive care becomes available. Such ready-to-use, storage-stable FS preparation(s) would become a valuable tool for emergency care providers, and on ambulances and rescue vehicles. As a result ready-to-use, storage-stable FS preparations will allow anyone to teat an injury victim, or even permit self-treatment, until medical assistance can be provided, making such a FS a valuable component of first-aid kits for the home, car, or office or on public transportation.

Ideally, the FS product should require as little manipulation as possible in its preparation, to minimize risks and the burden on the assisting personnel. Currently, fibrinogen-based FS preparations require a fibrinogen component that is available only as a lyophilate, a deep-frozen concentrate, or as a mixture with other components that could negatively alter hemostasis, or its safe use with a human patient. Thus, until the present invention there has remained a. need for a ready-to-use FS composition that is rapidly prepared from a storage-stable, aqueous fibrinogen solution, which despite its high concentration, remains available in fluid form, permitting easy processing into the instantly-available FS product for use on humans or animals, and which is both safe and effective, without risk of adverse effects.

### SUMMARY OF THE INVENTION

The present invention provides supplemented and unsupplemented, ready-to-use and instantly-available fibrin sealants (“FS”), prepared from ready-to-use, storage-stable, concentrated liquid fibrinogen preparations. The thus-produced FS product when applied to a tissue provides the elasticity, tensile strength, and adhesiveness necessary to prevent blood loss, to promote wound healing, and for many other therapeutic and non-therapeutic applications. It further provides methods of preparation of the supplemented and unsupplemented, storage-stable FS products of the present invention, and methods of use therefor.

The supplemented and unsupplemented, storage-stable FS products of the present invention are unique in that they are advantageously instantly available in ready-to-use form because the components used in their preparation are storage-stable and ready-to-use. Specifically, the fibrinogen component of the present FS is biocompatible, and remains available in fluid form at appropriate concentrations to permit rapid and easy preparation of FS. The sterile, storage-stable fibrinogen is aqueous and fully solubilized, its stability is pH and temperature dependent, and it retains its biological activity (i.e., the ability to rapidly form a fibrin clot upon exposure and vigorous mixing with thrombin and calcium ions). The thus prepared and stored, ready-to-use, concentrated human fibrinogen solutions may be neutralized and used without additional steps or processes in the preparation of biocompatible instantly available FS compositions.

One of the benefits of fibrin sealants is the natural bioabsorption that occurs after the cross-linked fibrin product has sealed the wound. This action, resulting from plasmin mediated lysis, permits natural removal of the fibrin sealant from the body and provides methods for accelerated removal if needed. This property, along with the superior adhesiveness and elasticity of the FS product, contributes to the value and versatility of the FS products as an emergency treatment that can be processed by hospital personnel once the patient is received or as an adjunct to surgical procedures. The FS product may advantageously be used directly on open wounds, or its use may be combined with other bandaging or suturing systems.

It is therefore an object of the present invention to provide a ready-to-use FS composition which can rapidly form a strong, yet flexible biologically compatible bond between separated tissues, or to achieve a coating or seal of a wound or undesirable opening in a tissue, to apply a graft, to coat a prosthetic material, or to deliver additive compounds to the surrounding tissue or circulatory systems. It is preferred that the resulting bond, covering or seal be watertight. Such composition is effective for its intended purpose on tissue in vitro, as well as in vivo in a human or animal patient. Further it is an object of the invention to provide FS compositions in which viscosity and/or polymerization time can be modified according to the desired application to facilitate placement of the composition at the tissue site.

It is still another object of the invention to provide a method of bonding separated tissues, of sealing or coating a wound or tissues to form a watertight seal, of applying a graft, or of coating a prosthetic material using a FS composition which is easy to handle, particularly during surgical procedures.

It is yet another object of the invention to provide methods of formulating such FS products, as well as methods of using same in vitar or in vivo to seal wounds, to apply grafts, to coat prostheses, or to deliver additive compounds to the surrounding tissue or circulatory systems.

It is also an object of the invention to provide methods of applying the instant FS components and the final FS product to the tissue or wound site. A particular advantage of the invention is the ready-to-use availability of the components, including the storage-stable fibrinogen component as an aqueous solution. Thus, the components are combined either immediately before application to the tissue or wound site or simultaneously with application to form the FS product. Dual syringe delivery devices can be easily used with consistent results using the described components of the instant FS composition because the components are stored in ready-to-use condition, and may, in fact, be separately and stabily stored within the barrels of the syringe for ease of delivery.

In the alternative, the components may be separately and stabily stored in divided compartments within a single barrel syringe, such that an affirmative action, such as pressing the plunger will cause the barrier to open permitting the pre-measured components to mix. Delivery of the FS composition is then instantly directed from a single port or needle to the tissue or wound site allowing the polymerization and cross-linking of the fibrin clot to occur directly at the site. In yet another alternative, syringe devices (single or multi-barrel) may be used to draw the storage-stable components from larger storage containers using standard syringe techniques, and the components or the mixed FS composition is delivered as described above, so long as fibrin polymerization and cross-linking occurs at the wound site.

It is a further object to provide kits for the ready-to-use delivery of an instant FS composition comprising at least two vials. One vial contains an aqueous solution of storage-stable fibrinogen at a concentration suitable for forming FS when mixed with an activator solution, such as activated thrombin or a thrombin-like composition, and a second vial contains the activator solution (preferably thrombin) at a concentration suitable for forming FS when mixed with the contents of the storage-stable fibrinogen in the first vial. CaCl₂ is added to and stored with the contents of one of the at least two vials in an effective amount to ensure fibrin polymerization, or in the alternative, the CaCl₂ component is supplied in an additional vial. Additional components, such as a stabilizer and/or Factor XIII and/or additives, such as a growth factor, drag, antibiotic, and the like are supplied by one or more additional vials, or alternately such additional components are added to and stored with the contents of the at least two vials.

Notably, however, a vial of The kits herein provided is expressly intended to also include a barrel of a syringe device. Accordingly, in one embodiment, the kit includes the described components provided in a single divided barrel or multi-barrel syringe device, so long as the fibrinogen component and the activator component remain separated until the instant FS composition is mixed and delivered.

Additional objects, advantages and novel features of the invention will be set forth in part in the description, examples and figures which follow, and in part will become apparent to those skilled in the art on examination of the following, or may be learned by practice of the invention.

### DETAILED DESCRIPTION OF PREFERRED EMBODIMENTS

The invention comprises supplemented and unsupplemented, storage-stable fibrin sealants (“FS”), prepared from ready-to-use, storage-stable, concentrated liquid fibrinogen solutions. The present FS is novel because it is instantly available since the components therefor are storage-stable and ready-to-use. In particular, the fibrinogen component is a shelf-ready aqueous solution, which is “storage-stable,” that is, after a period of days, weeks, months or longer, it remains stable in liquid form, it has not spontaneously clotted (i.e., it has not formed a “spontaneous clot,” even in the absence of an activator, such as thrombin/Ca⁺⁺), and it retains its biological activity (i.e., the ability to rapidly form a fibrin clot upon exposure and vigorous mixing with thrombin and Ca⁺⁺). The disclosed methods, therefore, set forth the conditions under which the FS components, including fibrinogen, is stored in a ready-to-use, aqueous solution for a period of days and remains active and stable (storage-stable).

The FS composition of the present invention is noninfectious, and provides a tissue bond having high tensile strength, elasticity, deformability, water tightness, viscosity and adhesivity for a large variety of surgical procedures. The composition can also be used to coat implantable devices to enhance their strength and resistance to fluids, to seal pores in the weave of a material implant device, and to reduce thrombogenicity. In the present disclosure, unless defined otherwise, all technical and scientific terms used herein have the same meaning as is commonly understood by one of ordinary skill in the art to which the invention pertains.

The FS composition of the subject invention comprises a fibrin polymer prepared from any form of fibrin monomer. In a preferred embodiment of the present invention, the FS fibrin polymer forms “instantly” or within seconds of activation of the storage-stable fibrinogen component of the preparation.

The enzymatic conversion of fibrinogen to fibrin is a two-step process. First, activated thrombin or a thrombin-like molecule cleaves the outer A and B terminal peptides of the fibrinogen molecule to form a soluble monomer, fibrin I, that is susceptible to internal catalysis. Activated Factor XIII (which is up-regulated by the conversion of prothrombin to thrombin) catalyzes the formation of amide bonds between a pair of amino acids in the fibrinogen monomers, resulting in the final cross-linked, insoluble fibrin II matrix. The cleavage only slightly reduces the molecular weight of fibrinogen from 340,000 daltons to only 334,000 daltons, but the process exposes the essential polymerization sites to permit formation of the assembled and cross-linked fibrin clot. See, Jackson. Ann. Rev. Biochem. 49:765-811 (1980); Furie et al, Cell 53:505-518 (1988).

The conversion of fibrinogen into fibrin via the enzymatic activities of activated thrombin and Factor XIII occurs under precise physiologic conditions. Exogenous generation of a matrix that possesses the superior clotting properties of natural fibrin requires that these requisite physiologic conditions be met. The FS of the present invention is in one embodiment is activated by the mixing of two activated or self-activating components at the time of delivery. The first component is a concentrated fibrinogen preparation, which in certain embodiments further comprises a protease inhibitor, such as aprotinin. This is mixed equally with the second activator component comprising thrombin or a thrombin-like equivalent and calcium, such as CaCl₂, although the second activator components maybe sufficiently available at the wold site that additional components are not needed. Each of the two components, and any compound added thereto, is selected and prepared to ensure that physiologic fibrinogenesis is duplicated as closely as possible in the FS product.

Nonlimiting examples of fibrin monomers include fibrin I monomer, fibrin II monomer or des BB fibrin monomer, or combinations thereof. Technically the term “FS composition” is used to refer to the mixture of the fibrinogen and activated thrombin or thrombin-like activator (as well as other essential and/or additive components) in the seconds before a fibrin clot forms. Once the FS fibrin clot has irreversibly formed, the term “FS product,” or simply “fibrin sealant” or “FS” are herein used. Nevertheless since the transition from FS composition to FS product occurs within only a few seconds, and since it is a continuous clot forming process, without a bright line change from “composition” to “product,” the terms are essentially interchangeable. More importantly, the terms are used to indicate the temporal transition from mixture of components in the FS composition to final FS clot in the resulting FS product.

Also, for the purpose of the subject invention, “fibrin polymer” includes any polymer resulting from the polymerization of fibrin monomer. Thus, for example, the conversion of fibrin I monomer to fibrin polymer can result in fibrin I polymer, cross-linked or noncrosslinked, and/or fibrin II polymer, cross-linked or non-cross-linked, depending on how the conversion step is carried out.

The viscosity of the components of the FS composition, as well as that of the FS fibrin preparation formed upon activation of the FS components, can be varied so that delivery, positioning, and stability during polymerization are appropriate to provide the necessary sealing capability, elasticity and strength for the selected FS application. Such attributes allow faster, more efficient surgical repair of damaged or weakened tissues than is possible with suturing or known sutureless procedures. The FS product, preferably delivered to the wound site in the form of a solution, most preferably as an aqueous solution, must provide the tensile strength necessary to keep the welded tissue together, joining the separated tissue or providing a watertight, flexible seal on a tissue, or prosthetic or implant surface.

Optionally, a viscosity modifier and/or bonding enhancer may be added as described below to the composition according to need. The resulting composition provides a FS product having excellent strength and superior handling characteristics. The composition is particularly suited for laser welding by forming a strong, uniform, elastic weld or coating.

The FS composition of the present invention comprises protein components selected from natural or synthetic peptides, including full-length molecules, enzymatically active modified, cleaved, or shortened variants thereof, or cross-linked derivatives thereof (Coller et al., J. Clin. Invest. 89:546-555 (1992)), as well as mixtures thereof. Included among the peptides are simple proteins, conjugated proteins, and mixtures thereof. Examples of such proteins include globular proteins and fibrous or structural proteins. Examples of globular proteins include synthetic or natural serum proteins, natural or synthetic derivatives thereof, salts, enzymatically, chemically, or otherwise modified, cleaved, shortened or cross-linked, oxidized or hydrolyzed derivatives or subunits thereof, and mixtures thereof.

The FS composition is prepared in a form ranging from a flowable liquid to a sol to a viscous gel depending upon the application and the concentration of components. For example, the composition is preferably employed in the form of a viscous gel for bonding separated tissues, wherein the gel quickly polymerizes into a durable, water insoluble, irreversibly cross-linked clot to secure the tissues together. On the other hand, the formation of a. watertight or resistant seal on tissues or prosthetic materials may be most efficiently accomplished using a less viscous composition. In some cases activation of the storage-stable fibrinogen component will spontaneously form a weld. In other cases, it may be necessary to activate the composition, with energy and/or photons.

Components of Instant Fibrin Sealant

Storage-Stable Fibrinogen Component

The characteristics if the FS composition of the present invention are defined and distinguished from prior art compositions that may be used for similar applications by the nature of the components from which the subject FS is prepared. The primary component of the preferred embodiment of the present FS invention is a highly concentrated solution of fibrinogen. Key to the present FS composition is its shelf-ready instant availability for its intended purposes, which is enabled by its formulation using a storage-stable, ready-to-use aqueous fibrinogen component, see e.g., U.S. patent application Ser. Nos. 10/267,104 and 10/263,987, the contents of which are herein incorporated by reference.

The storage-stable fibrinogen component may be originally prepared from any fibrinogen preparation, whether isolated and purified from blood plasma, produced by cell-culture techniques, recombinantly prepared, or freshly isolated, or freshly prepared from a lyophilized or deep-frozen plasma-derived preparation. Regardless of the source, the fibrinogen preparations are handled and used in essentially the same way once concentration and components remade equivalent. The storage stability of the fibrinogen component is irrelevant to the original source of the fibrinogen; in fact, it is the storage method and conditions of the aqueous solution that cause the fibrinogen solution to remain stable, while others have failed to create a suitable storage-stable fibrinogen solution.

After addition of thrombin/Ca⁺⁺ to the ready-to-use fibrinogen solution, the rapid increase in viscosity and decrease in liquid movement that is seen, is referred to as a “gel.” In the gel state, the fibrinogen solution no longer flows freely, but can be forced to move with agitation. Although this measurement is subjective, the estimated variability is only ±2 seconds.

“Clot” formation is the sudden solidification of the fibrinogen solution, beyond which agitation cannot force liquid to flow from the solidified material. The immobile material usually becomes macroscopically opaque white and viscoplastic. Scanning electron macrographs (SEM) photographs of typical physiological or non-physiological fibrin clots are shown, for example, in Redl et al., Medizinische Welt 35:769-76 (1985). A clot generally adheres to a test tube wall and cannot be dislodged by sharp tapping of the tube on a solid surface. This measurement is less subjective than gel formation, and estimated uncertainty is only ±1 second for rapidly setting samples (8-12 seconds), although it may be slightly greater for slower clotting (>100 seconds) samples.

Preparing the Fibrinogen Component

When the fibrinogen component is prepared from a lyophilized or deep-frozen plasma-derived preparation, the length of time the fibrinogen preparation has been lyophilized or deep-frozen is not a factor in the preparation of the FS composition of the present invention, so long as the biological activity of the freshly prepared fibrinogen solution is equivalent to a comparable sample of isolated and purified fibrinogen from fresh plasma, and spontaneous clotting has not been induced in the solution.

When the fibrinogen component is prepared from whole blood, typically a volume of blood, such as 100 ml, is collected into a standard commercially available blood bag containing an anticoagulant. Any anticoagulant can be used, such as, without limitation, heparin, EDTA, hirudin, citrate or any other agent that can, directly or indirectly, prevent the formation of thrombin. Citrate is preferred, and is commonly found in commercially available fibrinogen preparations. The plasma, which contains the fibrinogen component, is then separated from the whole blood.

Currently available, commercial fibrinogen contains salts used in the isolation and purification process. As noted in the Examples, this includes sodium citrate and sodium chloride, but presence of such salts that are a residual part of the fibrinogen purification process do not appear to affect the storage-stability of the resulting preparation or its effectiveness in the preparation of the present FS composition. Since the storage-stable, ready-to-use fibrinogen solution is only effective if it retains the characteristics of a comparable, freshly prepared fibrinogen solution, the effect of the fibrinogen purification process is essentially the same for both and not relevant to the present invention. Nevertheless, extremely high concentrations of citrate and/or sodium may affect clotting of the stored fibrinogen preparation.

Nonlimiting sources of FS components are blood, preferably mammalian blood and even more preferably human blood, cell cultures that secrete fibrinogen and recombinant fibrinogen, with blood plasma being preferred. Blood can be any form of blood including, for example, whole blood. Also, blood can be utilized to prepare an autologous fibrin sealant (from the patient's own blood products). Autologous fibrinogen can be prepared and stored for later use by the human or veterinary patient using the storage-stable methods of U.S. patent application Ser. Nos. 10/267,104 and 10/263,987.

Any separation technique can be utilized, for example, sedimentation, centrifugation or filtration. For example, using centrifugation, the blood is transferred to a container suitable for centrifugation and centrifuged at room temperature for 10 minutes at 3,000 g. The clear supernatant plasma (approximately 50 ml) is decanted and the cellular components are discarded. However, if it is desired to obtain plasma rich in platelets, centrifugation can be carried out at lower g force, e.g., 500 g for about 20 minutes. The supernatant, which contains the plasma, can be removed by standard techniques. Fibrinogen is then isolated from the resultant plasma and treated to preserve stability, e.g., in accordance with U.S. patent application Ser. Nos. 10/267,104 and 10/263,987, until needed to prepare the FS of the present invention.

In one embodiment, the plasma-derived fibrinogen component is prepared from whole blood by filtration. Filtration can be carried out by passing the whole blood through a suitable filter that separates blood cells from plasma. It is preferred that the filter be a microporous membrane exhibiting good protein transmission. As above, 100 ml whole blood is collected into a bag containing a suitable anticoagulant, then the blood is then recirculated over a filter exhibiting good protein transmission by means of peristaltic pump. The pressure drop across the membrane results in plasma being forced through, while cellular components remain in the recirculating blood. Plasma (50 ml) is collected for further processing as described above.

In an alternative embodiment, any cell culture that can secrete fibrinogen can be utilized in the subject invention. The culture and maintenance process is carried out essentially as described by standard texts on mammalian cell culture. For example, HEPG2 cells may be used for this purpose (see, e.g., Liu et al., Cytotechmology 5:129-139 (1991)). The cells are seeded into flasks at a split ratio between 1:4 to 1:8 in Minimal Essential Medium containing 10% calf serum and buffered with 5% CO₂ and maintained at about 37° C. After 24-36 hours the medium is removed and replaced with serum free medium containing a suitable protease inhibitor and 2 IU/ml heparin. Culture is continued for additional 24 hour periods, with three consecutive changes of serum free media. The conditioned media is centrifuged at 3,000 g for 10 minutes to remove any cell debris and the clarified supernatant contains fibrinogen, which can be further concentrated as desired using known methods.

The fibrinogen component of the present FS composition can also be prepared from recombinant DNA techniques (see, e.g.; Roy et al, J. Biol. Chem. 266:4758-4763 (1991)). Roy et al. teach methods for expressing all three chains of fibrinogen and teach that COS cells express, assemble and secrete the chains in a form that is capable of forming a thrombin-induced clot. Once prepared, the cellular debris is removed by centrifugation or filtration as described above as used for cell cultures, and then the fibrinogen may be concentrated.

The preparation by cell culture or recombinant techniques of the storage-stable fibrinogen component used in the present invention may be preferred in certain embodiments because viral contamination by plasma contaminants is eliminated, and there is more complete control over the presence of other components in the final FS. For example, Factor XIII is often present in fibrinogen preparations from plasma. However, unless expressly added, no Factor XIII is present in fibrinogen prepared by cell culture, permitting the amount added to the FS composition to cause cross-linking of the fibrin strands to be precisely quantified.

The preferred embodiments of the invention are applicable to a crude fibrinogen product in the course of preparation, or to a final, concentrated fibrinogen preparation having greater than 90% protein purity and being greater than 95% clottable protein, or to any concentration of fibrinogen there-between. For instance, in the Examples that follow, the human fibrinogen preparation had 53% protein purity and 95% clottable protein, while the bovine fibrinogen preparation had 61% protein purity and 97% clottable protein. Nevertheless, both were applicable to preparation of the FS composition of the present invention.

In a preferred embodiment of the invention, the storage-stable fibrinogen preparations of the present invention, although highly concentrated, remain solubilized in aqueous solution making the fibrinogen particularly suitable for use in the preparation of supplemented or unsupplemented, ready-to-use FS compositions. The fibrinogen is optimally stored at a concentration of 10-85 mg/ml, more preferably at a concentration of 15-75 mg/ml, even more preferably at a concentration of 30-70 mg/ml, and most preferably at a concentration of 40-65 mg/ml when is used to prepare a ready-to-use FS composition. Moreover, the concentration of fibrinogen, or fibrinogen-containing protein, in the storage-stable aqueous solution of the present invention generally ranges from 2 to 10 w/v %. preferably 4-7 w/v %. The concentration of fibrinogen is determined by protein absorbence measurements at 280 nm (using 14 as the extinction of 1% fibrinogen solution).

In the preferred embodiments of the invention, storage-stable fibrinogen is biologically active (i.e., clot in the presence of thrombin and Ca ions), and have essentially the same physical characteristics as fresh samples. This produces the same type of controlled fibrin clot formed using freshly prepared fibrinogen when FS compositions are prepared and used. Fibrinogen (and thrombin) concentrations dictate time to clot formation, clot strength, clot adhesion, and thus hemostasis. For the purposes of discussion, this type of clot is referred to herein simply as a “fibrin clot” to differentiate the process from a “spontaneous clot,” wherein the latter may occur in an unstable, concentrated fibrinogen solution, even absent thrombin or another activator.

However, the terms are used herein only for the purpose of distinguishing the FS compositions prepared from the storage-stable fibrinogen solutions in which the activity of the composition is quickly demonstrated byte rapid formation of a fibrin clot when equal amounts of the fibrinogen and thrombin/Ca⁺⁺ are vigorously mixed, from a spontaneous clot which is indicative of instability in the prior art fibrinogen solutions. The fact that prior art, aqueous solutions of fibrinogen are known to be highly unstable, and tend to spontaneously clot upon storage, makes the storage of fibrinogen in ready-to-use liquid form impractical for even a day or two using previously recognized methods.

In preferred embodiments of the invention, prior to its use, the storage-stable fibrinogen is stored in a polymer, plastic or plastic-based container, although more preferably the plastic container is polypropylene. Glass is not to be used to store fibrinogen or platelets because glass enhances spontaneous clot formation.

The fibrinogen solutions of the present invention are ideally suited for forming a physiological fibrin structure when exposed to an activator solution, and fibrin clots are rapidly formed. This is proven by mixing the stored fibrinogen solution with an equal volume of a thrombin/CaCl₂ solution (comprising, e.g., 2.5 units/mg fibrinogen (100 units/ml) thrombin and 3-6 mM excess CaCl₂ over citrate or other chelators that may be added to solutions), as set forth below. If the resulting clot demonstrates a physiological fibrin structure, it will have the typical, spatial branched fibril structure shown when clots are formed by the action of thrombin on freshly-prepared or freshly isolated and purified human fibrinogen under physiological conditions, i.e., at an ionic strength of approximately 0.15 and approximately neutral pH.

Prior experiments have proven, by continuous observation and testing, that the aqueous fibrinogen solutions of the invention under the preferred conditions remain stable (active and not spontaneously clotted) for at least 97 days at pH 6.3 to 8.0, when stored at room temperature (˜23° C.) or refrigerated (˜4° C.). In fact, the component has been shown to be stable for extremely long periods of time, as compared with known deep frozen or lyophilized preparations of the concentrated protein that have been maintained without a substantial loss of activity (i.e., fibrinogen/thrombin fibrin clots are still rapidly formed upon mixing), even years after the initial storage of the fibrinogen product. Thus, “long term storage” means storage of the fibrinogen solution, preferably human fibrinogen solution, in ready-to-use form under the presently disclosed conditions, without substantial loss of protein activity for at least 3 days, preferably for at least 3 weeks, more preferably for at least 10 weeks, more preferably still for at least 6 months, even more preferably for at least 1 year, and yet more preferably for at least 2 years (assuming it is frozen for ≧1 year, and then stored at ˜4° for ≧an additional year). Therefore, under optimal conditions the fibrinogen solution will remain stable for periods at least or greater than 2 years.

Although it is preferred to use “human” fibrinogen in FS applications in accordance with the methods of the present invention for a human patient, the use of stabily stored, ready-to-use, aqueous fibrinogen solutions from other species, most preferably species of other mammals, is also applicable. In fact, there appear to be no species compatibility issues associated with the use of the stored human fibrinogen with a mammalian species. For example, the subject human fibrinogen maybe used following storage in aqueous solution to prepare, e.g., a biologically compatible tissue adhesive preparation for use in or on any species of mammal. However, it is understood that an advantageous application of the present human fibrinogen preparation results from its ready-to-use applicability to human subjects.

Fibrinogen Storage Conditions: Temperature and pH

The optimal temperature and pH of the storage-stable fibrinogen component would be known in accordance with the present invention, or both could be rapidly determined, by one of ordinary skill in the art using known means. However, aqueous-based gels could also be used in the present invention, so long as such material permits the complete solubilization of the fibrinogen contained therein, and so long as the preparation is sufficiently fluid as to permit the rapid preparation of ready-to-use biological tissue adhesives or other applications following storage in accordance with the methods disclosed herein. A key to the present FS invention is the fact that the fibrinogen component is stored in ready-to-use fluid form. In its ready-to-use form, it is stored neither as a lyophilized preparation, nor is it in a deep frozen state.

The temperature of the solution during storage is not particularly restricted, so long as the fibrinogen contained therein remains stable (i.e., neither inactivated nor spontaneously clotted). The preferred temperature for storage of the fibrinogen solutions of the present invention ranges from 1° to 25° C., more preferably from about 4° to about 23° C. When refrigerated, the optimal temperature is about 4° C.±1° C., at which temperature the product has proven to be stable for at least 1 year (data not shown). When storage is at room temperature, the optimal temperature ranges from about 20° to 25° C., more preferably from about 22° to 24° C., most preferably the temperature is about 23°±1° C., at which temperature the product has proven to be stable for at least 3 months (data not shown). Moreover, previously frozen samples (for up to at least 1 year) have been subsequently stably stored at 4° C. for at least an additional year, making the product available for use herein in ready-to-use form for at least 2 years.

The pH value of the aqueous fibrinogen solution is preferably adjusted during storage to approximately pH 6.0 to 8.2, more preferably pH 6.2-8.0, even more preferably pH 6.3-7.5, and most preferably pH 6.5 to 7.36 and exemplified at pH 7.24 for bovine fibrinogen, and most preferably pH 6.32 to 7.13 for human fibrinogen.

The pH of the storage-stable fibrinogen solution is determined by the buffer in which it is stored. In a preferred embodiment of the invention storage-stable fibrinogen solutions are prepared in histidine buffer, although other recognized, physiologically acceptable buffers known to the art may be used to prepare the storage-stable fibrinogen, so long as the resulting pH of the fibrinogen solution remains within the proscribed range, such that its activity is maintained, but it remains free of spontaneous clotting. Suitable buffers, e.g., 0.1 M, include but are not limited to achieve the pH levels such as those that are noted: histidine, pH 6.0 or 7.2 to 7.24; Tris pH 8.16; glycine pH 9.3; or carbonate, pH 9.05 to 9.31 or pH 9.86 to 9.9.

The optimal pH for the storage of a particular fibrinogen solution depends in part upon the temperature at which the material is to be stored, as is shown in the Tables that accompany the Examples which follow. However, in light of the information provided herein, one of ordinary skill in the art would be able to select the optimal pH for the fibrinogen solution based upon the planned storage temperature and conditions, knowing that the determining factor is whether the protein contained therein remains stable (i.e., neither inactivated nor spontaneously clotted).

For example, ready-to-use human fibrinogen stored at room temperature (˜23° C.) is optimally maintained at pH 6.3 to 7.1, preferably at approximately pH 6.32 to retain the ability to rapidly form a clot when the stored preparation is neutralized and exposed to thrombin/Ca⁺⁺. When ready-to-use human fibrinogen is stored under refrigeration (˜4° C.) the optimal pH is also optimally maintained at pH 6.32 to 8.0, preferably at approximately pH 6.3 to 7.5 to retain the ability to rapidly form the FS clot when the stored preparation is neutralized and exposed to thrombin/Ca++(see Table 2).

Similarly, ready-to-use bovine fibrinogen stored at room temperature (˜23° C.) is optimally maintained at pH 6.5 to 9.0, preferably at approximately pH 6.5 to 8.2, to retain the ability to rapidly form a clot when the stored preparation is neutralized and exposed to thrombin/Ca⁺⁺. When ready-to-use bovine fibrinogen is stored under refrigeration (˜4° C.), the optimal pH is also optimally maintained at pH 6.5 to 9.0, preferably at approximately pH 6.5 to 8.2, more preferably at pH 6.5 to 7.07 to retain the ability to rapidly form a clot when the stored preparation is neutralized and exposed to thrombin/Ca⁺⁺ (see Tables 1 and 2).

Activator Component, e.g., Thrombin or Thrombin-Like Enzyme

The “activator” of the present invention is thrombin or a thrombin-like enzyme. A “Thrombin-like enzyme,” including thrombin, means any enzyme that can catalyze the formation of fibrin from fibrinogen. In addition to thrombin from mammalian, blood sources, preferably from human sources for use with human patients, the enzyme can be produced by cell culture or recombinant means and isolated as described below regarding the fibrinogen component. Bovine thrombin is conveniently and commercially available from a variety of sources, including Parke-Davis.

Thrombin acts as a catalyst for fibrinogen to provide fibrin, an insoluble polymer. Thrombin is present in the FS composition in an amount sufficient to catalyze polymerization of fibrinogen. Thrombin also activates Factor XIII, a plasma protein that catalyzes covalent cross-links in fibrin, rendering the resultant clot insoluble.

As an alternative to thrombin or thrombin analogs, a common source of thrombinlike enzymes are purified from the reptilase coagulants i.e., snake venoms (see, e.g., Pirkle et al, Thrombosis and Haemostasis, 65(4):444-450 (1991)). A preferred thrombin-like enzyme is, without intended limitation, ancrod or batroxobin, especially from B. Moojeni; B. Maranhao and B. atrox and Ancrod, especially from A. rhodostoma. Depending on the choice of thrombin-like enzyme, such thrombin-like enzyme can release fibrinopeptide A, which forms fibrin, although at different rates than thrombin.

It should be noted that if the storage-stable fibrinogen component of the present FS preparation comes into contact with the patient's blood, e.g., at the wound site, the patient's own thrombin and Factor XIII may be sufficient to convert the fibrin polymer to cross-linked fibrin polymer. Thus, endogenous prothrombin and Factor XIII can be utilized in the FS of the subject invention as components of the composition comprising fibrin monomer or non-cross-linked fibrin. However, it should be noted that sufficient quantities of endogenous thrombin and Factor XIII are typically not retained in amounts sufficient to convert the storage-stable fibrinogen component to cross-linked fibrin at a reaction rate that is suitable for producing an effective fibrin seal. In larger wounds, the heavier blood flow will wash away the endogenous material, and clotting will not take place. It appears that more thrombin is required to convert fibrinogen to cross-linked fibrin than to convert non-cross-linked fibrin to cross-linked fibrin at an equivalent reaction rate.

The concentration of the thrombin component in the FS composition of the present invention can range from as little as 150 μg thrombin per 40 mg fibrinogen in solution to an equal ratio of thrombin and fibrinogen in solution, depending on the application, surrounding conditions (e.g., temperature, pH, mixing), and the rate of polymerization desired. In terms of the FS composition rather than the fibrinogen component, from about 4 units to about 500 units of thrombin per ml of FS composition is added. Alternately, the thrombin component can be provided by the wound site. However, polymerization of the fibrinogen component will proceed more quickly as more thrombin is available to activate each molecule of fibrinogen in solution, up to a maximum at which point increased polymerization is not possible by the addition of thrombin alone.

A source of calcium ions, e.g., as CaCl₂, is essential to activate the thrombin component before the thrombin component can activate the fibrinogen component to for the present FS composition. However, the calcium ions may be incorporated into the stored thrombin component. Alternatively, CaCl₂ may be added to the FS composition prior to polymerization, or sufficient quantities of calcium ions may simply be endogenously available at the wound site.

In Factor XIII-free FS preparations, Factor XIII is optimally added to activate crosslinking of the fibrin. Activated Factor XIII can be added to the FS composition at a final concentration of from about 1.0 to about 20 units Factor XIII per ml of FS composition. Alternatively, the Factor XIII can be supplied by the blood or body fluids at the wound site, or by the addition of autologous plasma.

In one embodiment of the present invention, the activator enzyme is immobilized onto a support. This can be carried out by any suitable technique. For example, various activation chemistries available for derivatizing supports are: diazonium groups, isocyanate groups, acid chloride groups, acid anhydride groups, sulphonyl chloride groups, dinitro fluorophenyl groups, isothiocyanate groups, hydroxyl groups, amino groups, n-hydroxysuccinimide groups, triazine groups, hydrazino groups, carbodiimide groups, silane groups and cyanogen bromide. See e.g., Dean, in Affinity Chromatography—A practical Approach, Johnson and Middle (Eds) (1991) IRL Press Oxford, the procedures of which are incorporated by reference. Low pH values, e.g., pH 4-6, can be utilized for enzyme coupling to prevent enzyme degradation.

Agarose may be used as the support, although it is also possible to use silica. Generally, The support is activated by a highly reactive compound, which subsequently reacts with a functional group of a ligand, e.g., —OH, —NH₂, —SH, —COOH, —CHO, to form a covalent linkage.

In certain embodiments of the invention, the FS composition is activated through the application of energy and/or photons. The energy preferably has a wavelength in the electromagnetic spectrum, and is selected from X-rays, ultraviolet light, visible light, infrared light, and radiowaves. Thermal energy delivered through direct contact as, for example, with a probe heated electrically, such as an electrocautery, or a probe heated through gas compression in the tip, or the passage of heated gas or liquid through the tip, may be used. Sound energy in the ultrasonic frequency, or radiowaves in the microwave range may also be employed. The energy is delivered in a continuous or noncontinuous fashion, in a narrow or broad band of electromagnetic wavelengths. Examples of photon sources include monochromatic and polychmromatic light, coherent or noncoherent light, delivered in a continuous or noncontinuous fashion. Examples of noncontinuons energy and/or photon delivery include single and/or multiple pulse train delivery. Photons can be delivered in a polarized or nonpolarized fashion, direct or reflected, with or without internal or external interference. In a preferred embodiment, lasers are used, including, but not limited to, those in the ultraviolet, visible, or infrared range.

Stored solutions of ready-to-use human fibrinogen that do not clot when thrombin and calcium ions are added with vigorous agitation are called “thrombin-insensitive.” The thrombin insensitive preparations remain fluid (having viscosities similar to water). However, analysis of such thrombin insensitive fibrinogen samples by SDS-PAGE (sodium dodecyl sulfate polyacryamide gel electrophoresis) has shown that the fibrinogen protein has been irreversibly degraded to small molecular weight fragments. Thus, the preparation no longer contains active fibrinogen, and is not the subject of the present invention.

Supplements and Additives

As noted, the FS composition of the present invention can additionally contain viscosity modifiers and/or bonding enhancers in accordance with the end use of the composition. For example, the addition of viscosity modifiers provides a FS composition with a viscosity particularly suited to the tissues being repaired or sealed. A composition having a high viscosity is preferably employed to bond separated tissues while lower viscosity compositions are best suited to form a coating for watertight sealing of continuous tissue masses and prosthetic materials, such as Gortex™ vascular grafts and the like. Such viscosity modifiers include, without limitation, non-cellular matrix materials, such as hyaluronic acid and salts thereof (e.g., sodium hyaluronate or sodium chondroitin); or saccharides, such as fructose, hydroxypropyl-methylcellulose, hydroxyethylcellulose, carboxymethylcellulose, hydroxymethylcellulose, dextrans, agarose, alginic acid or pectins; or polyalcohols, such as glycerin; or protein gels, such as collagen and caseinates; or mixtures thereof.

Bonding enhancers may also be used to improve the bonding strength of the composition. Such bond enhancers may be (i) added to the activator component before mixing with the storage-stable fibrinogen component or (ii) added to the activated fibrinogen/fibrin mixture prior to polymerization, or (iii) spread over the wound surface prior to application of the FS material. The bond enhancers are generally selected from polar compounds, such as charged glycosaminoglycans, oligosaccharides and polysaccharides, polyalcohols, and polar dyes. Notably many of these compounds also operate as viscosity modifiers. Examples of such polar compounds include without limitation, hyaluronic acid, chondroitin sulfate, carboxymethyl-cellulose, hydroxymethylcellulose, glycerin, indocyanine green, and fluorescein sodium. Polyvalent cations, such as calcium, may also enhance bonding by binding to the negatively charged moieties in the protein components of the FS composition, such as albumin, and glycosaminoglycans, such as hyaluronic acid and chondroitin sulfate.

Mucoadhesives are particularly useflul bond enhancers when the wound surface contains mucin, such as the gastrointestinal tract and the pulmonary system. Examples of mucoadhesives include carboxymethylcellulose and sodium alginate. Use of these materials on wound surfaces having a high collagen content, which have a large concentration of hydroxyl groups, may also be advantageous in facilitating bond formation. As reported by Robinson et al., Ann. NY Acad. Sci. 507:307-314 (1987)), a high charge density is preferred for both swelling and hydrogen bonding, thus permitting firm attachment to the desired tissue surface. Other mucoadhesives as would be obvious to one skilled in the art may also be employed.

The composition may as necessary additionally contain pH modifiers, surfactants, antioxidants, osmotic agents, and preservatives. Examples of pH modifiers include, for example, without limitation, acetic acid, boric acid, hydrochloric acid, sodium acetate, sodium bisulfate, sodium borate, sodium carbonate, sodium citrate, sodium hydroxide, sodium nitrate, sodium phosphate, sodium sulfite, and sulfuric acid. Examples of surfactants include, for example, benzalkonium chloride. Examples of antioxidants include, for example, bisulfates. Examples of osmotic agents include, for example, sodium chloride. Examples of preservatives include, for example, chlorobutanol, sorbate, benzalkonium chloride, thimerosal, methylparaben, propylparaben, EDTA (ethylenediaminetetraaetic acid), and polyquad.

Typically, pH modifiers, surfactants, antioxidants, osmotic agents, and preservatives are present in a concentration of from about 0.001 to 5% by weight.

The components of the composition are combined together in quantities, which provide a desired bonding strength, as well as a viscosity, which is particularly adapted to the intended end use. In general, the amount of the peptide in the FS composition is in the range of from about 1 to 99% by weight preferably about 5 to 80% by weight, more preferably about 6 to 70% by weight, more preferably still about 6 to 50% by weight to about 8 to 35% by weight. Saccharides, if present, may be combined in the range of from about 0.1 to 70% by weight. Glycosaminoglycans, if present, is preferably from about 0.1 to 20% by weight. Polyalcohols, if present, may be added in an amount of from about 0.1 to 90% by weight.

The amount of additives, such as viscosity modifiers and bonding enhancers is generally no more than about 65% by weight.

The viscosity of the FS composition is chosen in accordance with the particular surgical procedure being performed. For bonding of separated tissues, a viscosity of from about 1,000 to 1,000,000 centipoise is advantageous, preferably in the range of from about 20,000 to 200,000 centipoise. A FS composition having a viscosity in the preferred range can be easily placed on the separated tissues by ejecting through a hypodermic syringe or dual-syringe device, and spreading over the wound area by moving the syringe tip. Within that viscosity range, the FS composition does not run off the tissues and remains fixed, even when energy is applied to form the tissue weld.

The viscosity of the FS composition or the present invention is preferably lower for applications requiring the formation of a watertight coating for sealing tissues or prosthetic materials. For such purposes, the preferred viscosity for coating is in the range of from 10 to 1,000 centipoise. The lower viscosity is preferred to permit the ready capability to spread the composition to efficiently cover the tissue or material being coated.

When hyaluronic acid, or other non-Newtonian fluids are added to the FS composition, the viscosity decreases with increasing shear forces. Accordingly, the viscosity of the FS composition can be modulated by altering the shear forces when the composition is applied to the wound surface. For example, a very thick FS composition can be injected through a graft at a rapid or high sheer rate to reduce viscosity during the transit phase in which the graft is coated with the material applying a property known as pseudoplasticity, This makes the highly viscous FS material ideal for welding at sites that are not subject to shearing forces during the polymerization process. When the composition is injected, shear forces are high, and viscosity decreases, permitting easy injection. After being deposited on the tissue, the shear forces drop to zero, and the viscosity of the composition rapidly increases accordingly. As a result, the composition remains localized on the tissue.

In certain embodiments of the present invention, the FS composition is supplemented with, and acts as a carrier vehicle or delivery vehicle for, any number of compounds, alone or in combinations of two or more, for example, but not limited to, growth factors, drugs, blood factors or other compound(s) or mixtures thereof, so long as noted above, the activity of the fibrinogen solution is maintained throughout the length of the storage and spontaneous clotting is not induced. For instance, by supplementing the FS composition, or one component of the FS composition, such as the storage-stable fibrinogen component with a growth factor, the FS composition can, when applied to a human patient or animal subject particularly at a wound site, accelerate, promote or improve wound healing, tissue (re)generation, and the like.

The supplement may be mixed with the fibrinogen or thrombin component, or with a combination thereof, or with the final FS composition, depending on the nature of the additive, the FS polymerization rate, interaction between components, and the like. It is believed that the dosage of such supplements is the same as that utilized in conventional fibrin sealants.

Optimally, the supplemented FS composition when used as a carrier or delivery vehicle acts to: (1) potentiate, stimulate or mediate the biological activity of the growth factor(s), ding(s), or other additive(s) or component(s); (2) decrease the activities of one or more additive(s) or component(s) of the supplemented FS composition or storage-stable fibrinogen component used therein, wherein such activities would otherwise inhibit or destroy the growth factor(s) in the preparation; (3) allow prolonged delivery of the additive or component from the FS composition or the storage-stable fibrinogen component of the present invention; and (4) possess other desirable properties. The contemplated additive(s) or supplement(s) are intended to also include any mutants, derivatives, truncated or other modified forms thereof, which possess similar biological activity(ies), or a subset thereof, to those of the compound or composition from which it is derived.

More than one additive or component may be simultaneously added to or supplied by the FS composition of the present invention. Although the concentration of such additive(s) and/or component(s) will vary in the FS composition depending on the objective, the concentration must be sufficient to allow such compound(s) and/or composition(s) to accomplish their intended or stated purpose. The amount of such supplement(s) to be added can be empirically determined by one of ordinary skill in the art by testing various concentrations and selecting that which is effective for the intended purpose and site of application. Dyes, tracers, markers and the like may also be added, for example, to examine the subsequent delivery of the FS composition.

Supplemented FS preparation may comprise, e.g., drug(s), antibody(ies), anticoagulant(s), coagulation factors such as Factors VII, VIII, IX, X and XIII, and von Willebrand's factor, as well as growth factors, and/or other compounds that are presently delivered to a human or animal patient in need of such by other delivery devices or mechanisms that may not operate as efficiently or effectively as the present invention.

Various components may be added which serve to recruit or expand the leukocyte or endothelial population, inhibit pathways of leukocytes, endothelial cells or the like, or to modulate novel peptides. Compounds of biological value include, without limitation, growth factors, e.g. EGF, TGFα, TGFβ, TGF-I and TGF-II, FGF, PDGF, etc.; cytokines, e.g., IFN-α, IFNβ, IL-2, IL-2, IL-3, IL-6, hematopoietic factor, etc.; immunoglobulins; metabolic substances, e.g., insulin, corticosteriods, hormones, etc. Other materials include structural materials, such as physiologically acceptable alloplastic materials, e.g., polymers, glasses, metals, ceramics, composites thereof, etc.

Other materials can also be added, for example, fibronectin, fibrinolytic inhibitors, such as aprotinin, alpha-2 antiplasmin, PAI-1, PAI-2,6-aminohexanoic acid, 4-aminomethyl cyclohexanoic acid, or collagen.

The FS material may be mixed with cells, autologous, cultured or modified, allogeneic or xenogeneic, such as epithelial, epidermal, fibroblast, osteoblast, mesenchymal, hepatic (hepatocytes), pancreatic (e.g., macrophage, platelet, T-cell, B-cell, granulocytes, monocytes, keratinocytes, etc.), or cultured modified cells, to deliver therapeutic or growth enhancing substances.

For dental or orthopedic applications, inorganic minerals or a mixture of inorganic minerals, naturally occurring or synthetic, desirably hydroxyapatite or minerals found in bone powder or chips may be added to the formulation. The mineral(s) are present in a volume ratio to the fibrinogen component of from about 1:2 to about 4:1 depending upon the desired flow characteristics or intended use and site. Demineralized bone matrix (DBM) is a source of osteoinductive proteins, known as bone morphogenetic proteins (BMP), including osteogenin, and growth factors which modulate the proliferation of progenitor bone cells (see, e.g., Hauschka et al., J. Biol. Chem. 261:12665-12674 (1986) and Canalis et al., J. Clin. Invest. 81:277-281 (1988)). Unfortunately, DBM materials have little clinical use unless combined with particulate marrow autografts, and there is a limit to the quantity of DBM that can be surgically placed into a recipient's bone to produce a therapeutic effect. In addition, DBM powder and osteogenin may be washed away by tissue fluids before their osteoinductive potential is expressed. Moreover, seepage of tissue fluids into DBM-packed bone cavities or soft-tissue collapse into the wound bed are two factors that may significantly affect the osteoinductive properties of DBM and osteogenin. Soft-tissue collapse into the wound bed may likewise inhibit the proper migration of osteocompetent stem cells into the wound bed.

FS also can serve as a “scaffold,” which cells can use to move into a wounded area to generate new tissues. Additionally, viable osteoblasts may be harvested from a donor site and incorporated into the composition for use in transplantation. Other bone restorative materials in particulate form may be used. Among the suitable alloplastic materials are polylactic and polyglycolic acids, polymethycrylate, polyHEMA, bioglass, cerevital and other glasses, Al, Ti, CoCr and other metals, Al₂O₃ and other ceramics, etc., and combinations and composites thereof. They may be used in the same volume to volume ratios as for bone mineral. Other restorative materials, such as proteinaceous particles or beads made from collagen, fibrin, fibrinogen, albumin, etc., may be used as well, depending upon the tissue repair site. Liposomes may also be incorporated.

As previously noted the FS composition may additionally contain an antibiotic to reduce or prevent infection, e.g., gentamycin, cefotaxim, nebacetin and sisomicin, histaminine H₂-antagonists, e.g., ranitidine, and anticancer drugs (see, e.g., Gersdorff et al., Laryngoscope 95:1278-80 (1985); Ederle et al., Ital. J. Gastroenterol. 23:354-56 (1991); Ronfard et al., Burns 17:181-84(1991); Sakurai et al., J. Control. Release 18: 39-43 (1992); Monden et al., Cancer, 69:636-42 (1992); Greco, J. Biomed. Materials Res. 25:39-51 (1991.); Kram et al., J. Surgical Res. 50:175-178 (1991)). The antibiotic may be incorporated into a liquid component of the FS or into the resulting FS composition prior to polymerization, if the antibiotic is a liquid, or suspended in the liquid component, if it is in powder form. The therapeutic dose levels of a wide variety of antibiotics for use in drug release systems are well known (see e.g., Biomaterials, G. D. Winter, D. F. Gibbons, H. Plank (Eds.), John Wiley & Sons, New York (1980), pp. 669-676). Anti-microbial agents are particularly useful for compositions applied to exposed wound repair sites, such as sites in the mouth, or to compromised wound sites, such as burns.

Chromophores and Indicator Compositions

In one embodiment, the composition of the present invention further includes endogenous or exogenous chromophores to facilitate visualization of the material during placement into warm blooded animals. Use of a chromophore allows visualization of the FS for targeting to the wound site. It also provides a rapid means for identifying any material that is displaced from the desired application site, and permits subsequent removal of the extraneous material using a cellulose sponge, gauze pad, or other absorbing material. The use of endogenous chromophores, such as hemoglobin, is disclosed in Krueger et al, Lasers Surg. Med. 5:55-60 (1985)). The use of exogenous chromophores to aid in the placement of biological adhesives has been previously described (see, e.g., Nasaduke et al., Ann. Ophth. 18:324-327 (1986)).

Chromophores that may be used, include, but are not limited to fluorescein isothiocyanate, indocyanine green, silver compounds such as silver nitrate, rose bengal, nile blue and Evans Blue, Q-Switch™ (Kodak, Inc.), Sudan III, Sudan Black B and India Ink. The chromophores are preferably present in a concentration of from about 0.01 to 50% by weight based on the total weight of the composition. Other chromophores of types obvious to one skilled in the art may also be employed.

Such substances may also alter absorption characteristics of the composition so that the composition absorbs energy at low energy levels. This enables the heating of the material using certain wavelengths of the electromagnetic spectrum which are selectively absorbed by the energy absorbing compound. For example, this would allow heating of the material using certain lasers whose energy would otherwise not be absorbed by the composition of the present invention, and allows the composition to be bonded to the target using these lasers. The selection of dyes having a peak light absorption at a specific wavelength and matching tat to the wavelength of light emitted from a light source, such as a laser beam, allows for the selective activation of the composition at the site of the coating or seal, while substantially reducing the risk of undesirable collateral thermal damage to adjacent tissues.

Exogenous dyes, such as indocyanine green or fluorescein, and endogenous chromophores, such as hemoglobin and melanin, and the like, are particularly suited for this purpose. These dyes also may increase adhesivity, bond strength and/or viscosity. Such dyes are preferably present in the composition in an amount of from about 0.01 to 50% by weight based on the total weight of the composition.

Chaotropic Agents

In the event that it is desirable to delay formation of the FS product fibrin, a chaotropic agent is added to prevent spontaneous polymerization of the fibrin monomer, which is formed upon contact of the fibrinogen with the activator. The chaotropic agent is mixed with such fibrinogen composition and then agitated for about 1 to 2 minutes to form the modified fibrinogen solution. The fibrinogen can then be converted to a fibrin monomer as described above, but polymerization will be delayed.

Suitable chaotropic agents include, for example, urea, sodium bromide, guanidine hydrochloride, KCNS, potassium iodide and potassium bromide. The preferred concentration of the chaotropic agent is from about 0.2 to about 6.0 molar and most preferably from about 0.3 to about 2.0 molar. It is preferred to utilize the least amount of chaotropic agent possible that still prevents the fibrin monomer from spontaneously polymerizing. More preferably a source of calcium ions should not be added to the chaotropic agent until polymerization of the fibrin monomer is desired. This ensures that the fibrin monomer will not cross-link due to activation by endogenous blood coagulation factors.

If the chaotropic agent was added to the aqueous buffet of the fibrinogen or thrombin components, then the resulting fibrin composition can be converted to cross-linked fibrin by diluting the composition with, for example, distilled water. The dilution is carried out, such that the minimal amount of diluent is utilized. Generally, the resulting concentration of the chaotropic agent after dilution should be from about 0.5 to about 0.1 molar.

Buffering the FS Composition

Upon application to the wound site the FS composition is in one embodiment preferably buffered to acidity using an acid buffer having a pH of less than about 5. Nonlimiting examples of suitable acidic buffer solutions include acetic acid, succinic acid, glucuronic acid, cysteic acid, crotonic acid, itaconic acid, glutamic acid, formic acid, aspartic acid, adipic acid and salts thereof. Succinic acid, aspartic acid, adipic acid and salts of acetic acid are preferred, and sodium acetate is more preferred. The preferred concentration of the acid buffer ranges from about 0.02 M to about 1 M, more preferably from about 0.1 M to about 0.3 M. Such preferred concentration renders the ionic strength of the composition more biologically compatible.

Accordingly, in one embodiment of the present invention, the composition comprising fibrin monomer is substantially free of the activator enzyme. By “substantially free” is meant either that all of the thrombin or thrombin-like enzyme has been removed, or that any thrombin-like enzyme remaining in the composition is at levels insufficient to provide an undesired pharmacological effect. Thus, compositions of this invention that are substantially free may contain an activator enzyme in an amount between about zero and 10% of the enzyme normally found in a fibrin clot, and preferably between about zero and 2% of the enzyme.

A preferred embodiment of the present invention further provides methods of preparing the subject instant FS composition in accordance with the preceding definition.

In yet another preferred embodiment, the FS composition of the invention is prepared using a suitable alkaline buffer. Nonlimiting examples of suitable alkaline buffers include HEPES, sodium hydroxide, potassium hydroxide, calcium hydroxide, bicarbonate buffers such as sodium bicarbonate and potassium bicarbonate, tri-metal salts of citric acid, salts of acetic acid and salts of sulfuric acid. Preferred alkaline buffers include: 0.5.-0.75M sodium carbonate/bicarbonate pH 10-10.5, 0.5-0.75M sodium bicarbonate/NaOH pH 10.0, 1.5M glycine/NaOH pH 10.0, 0.5-1.0 M bis hydroxeythylaminoethane sulphonic acid (BES) pH 7.5, 1M hydroxyethylpiperazine propane sulphonic acid (EPPS) pH 8.5, 0.5 M tricine pH 8.5, 1M morpholino propane sulphonic acid (MOPS) pH 8.0, 1M trishydroxymethyl aminoethane sulphonic acid (TES) pH 8.0 and 0.5M cyclohexylaminoethane sulphonic acid (CHES) pH 10.0; with 0.5-0.75M sodium carbonate/bicarbonate pH 10-10.5, 0.5-1.0M bis hydroxeythylaminoethane sulphonic acid (BES) pH 7.5, 1M hydroxyethylpiperazine propane sulphonic acid (EPPS) pH 8.5 and 1M trishydroxymethyl aminoethane sulphonic acid (TES) pH 8.0 being most preferred.

The amount of alkaline buffer that is utilized should be enough to polymerize the fibrin. It is preferred that about 10 parts to about one part of composition comprising fibrin monomer be mixed with about 1 part alkaline buffer. It is even more preferred that such ratio be about 9:1, although the preferred ratio depends on the choice of buffer and the desired “strength” of the fibrin polymer. Of course, the desired strength of the fibrin polymer is determined by the intended end-use of the FS composition.

Activating FS Polymerization

In addition to raising the pH or diluting the chaotropic agent of the composition comprising fibrin monomer, it is preferred that the prothrombin and Factor XIII of such composition be activated to form the cross-linked fibrin. Such activation can be carried out by the contacting the components of the FS composition with a source of calcium ions. The source of the calcium ions can be included with the fibrinogen or thrombin buffer. As noted above, nonlimiting sources of calcium ions include calcium chloride, preferably at a concentration of about 30 mM. Alternatively, although less preferred, the source of calcium ions can be supplied by the blood at the wound site.

If a carbonate/bicarbonate buffer is used, the source of calcium ions must be added to the acid buffer during the solubilization step. This is because calcium chloride is not soluble in a carbonate/bicarbonate buffer. Preferably, the concentration of calcium ions in the acid buffer solution is from about 5 millimolar to about 150 millimolar, and more preferably from about 5 mM to about 50 mM.

Product Safety

Unless prepared by cell culture, FS compositions comprise blood plasma proteins, and as a result are accompanied by a risk of contamination with blood-borne pathogens, such as those possibly contaminating human plasma proteins, in particular, hepatitis viruses or HIV. Using known viral inactivation methods there have been no reports of viral transmission from commercial fibrin sealants, even when used on large bleeding surfaces. In the manufacture of plasma derivatives from pooled human plasma, viruses become partitioned as part of the fractionation process. Because specific viruses partition with some fractions but not others, in certain cases partitioning alone may be sufficient to clear a plasma derivative of a particular infective agent. However, from the AIDS epidemic, it is now known that while HIV may be effectively cleared from immunoglobulin, it can remain in antihemophilic factor concentrates. It is, therefore, of great importance that all plasma fractions are assumed to be contaminated and that vigorous inactivation methods be employed.

A number of viral inactivation strategies have been investigated and are described in the prior art literature. For example viral inactivation methods in blood products, include, but are not limited to dialysis, ultrafiltration, two-step vapor heating (cumulative), high temperature and pressure sterilization, solvent detergents such as tri(n-butyl) phosphate (TNBP) or Tween 80, photochemicals such as psoralen analogs, pasteurization (heating), radiation exposure, and ultraviolet light treatment. Although virus inactivation by high heating or steam methods are impractical for biologically active protein solutions, including the present fibrinogen solutions, nanofiltration is an optional treatment without causing inactivation of the components, such as human fibrinogen solution, of the present invention before placing it into the sterile storage container.

Viral inactivation methods that reduce infectivity by 5 logs should provide assurance that a preparation is no longer infectious. For instance, the vapor heating process used in the production of Tisseel VH fibrin sealant has been shown to reduce viral titers by at least 6.4 log reduction units for each vapor heating step of the two step process, bringing the risk of contamination to negligible. Various washing steps can be employed to remove stabilizing additives by methods known in the art. Methods known to effectively offer viral inactivation in to prior art fibrin sealant compositions may be used and will be equally effective for the instant FS products of the present invention.

Methods of Preparing and Using the FS Composition

The method of formulating the FS composition of the present invention may be performed in a number of ways, including, but not limited to the following preparation techniques, which generally result in a well formulated composition. The preparation is generally conducted at no more than 22.5°-30° C. Initially, the fibrinogen and the activator components are formulated into sterile aqueous solutions at the desired concentration. The advantage of the present invention that has not previously been possible is that the aqueous solutions can then be stabily stored for days, weeks or months without significant change in activity or ability to form the FS composition of the present invention when combined. When the FS composition is needed, the fibrinogen component is neutralized and combined with the activator component, which may also contain CaCl₂, Factor XIII and other additives, depending on the intended purpose of the FS. The components are combined in a ratio, which is determined by the intended end-use of the composition. To improve the mixing of the molecules of the primary components, it is generally advantageous to agitate the composition either internally, or externally, typically by stirring or shaking vigorously as described in the following Examples, until a sol or gel forms.

In alternative embodiments, additives to enhance viscosity, the bond, or visualization of the material may be added after the components are combined. Other components, such as pH modifiers, stabilizers, protease inhibitors, surfactants, antioxidants, osmotic agents, preservatives and the like may be added at this time, as well as components which do not affect the FS per se, but are added for delivery to the patient or tissues in vitro or in vivo.

In a preferred embodiment of the present invention, no antimicrobial agent is added to the fibrinogen, rather sterility is preserved using known techniques. However, in an alternative embodiment, antimicrobial agents are added to the extent exemplified, to avoid microbial contamination of the fibrinogen solution component over long term storage. Any recognized, physiologically antimicrobial agent is acceptable for the purposes of the present invention, so long as the activity of the fibrinogen solution is maintained throughout the length of the storage, spontaneous clotting is not induced, and the agent is not contra-indicated for human use.

The instant fibrin sealant of the present invention, prepared from storage-stable fibrinogen components, such as human fibrinogen, may be thus used in any known manner in which such biologically-prepared, supplemented or unsupplemented tissue adhesives are used, e.g., pharmacological or cosmetic uses, including for infusion purposes, such as delivery of antibiotics, antineoplastics, anesthetics, and the like; as a soft tissue augmentor or soft tissue substitute in plastic reconstructive surgery; to attach skin grafts to a recipient site without the use of sutures or with a reduced number of sutures, or as a growth matrix for transplanted intact osteoblasts in bone repair and reconstruction. The FS can also be used for applications such as ossicular reconstruction, nerve anastomosis or other situations where repair by sutures is impossible or undesirable, or as a wound dressing.

The FS may be applied in a number of ways determined by the surgical indication and technique for wound healing, coagulation and fibrinogenanemia; for inhibition of operative or post-operative sequelae; for coating prostheses; for dressable wound sealings and for safe and sustained hemostasis, namely sealing fluid and/or air leakage, and the like in a patient. Certain preferred embodiments of the invention provide methods of directly using the subject instant FS composition for connecting tissues or organs, for example, without limitation, to stop bleeding, heal wounds, seal a surgical wound, use in vascular surgery, include providing hemostasis for stitch hole bleeding of distal coronary artery anastomoses, left ventricular suture lines, aortotomy and cannulation sites, diffuse epimyocardial bleeding as occurs in reoperations, and oozing from venous bleeding sites. The subject invention is also useful for sealing Dacron and other grafts prior to insertion and for coating prostheses, stopping bleeding in spleens livers, and other parenchymatous organs, sealing tracheal and bronchial anastomoses and air leeks or lacerations of the lung, sealing bronchial stumps, bronchial fistulas and esophageal fistulas; for sutureless seamless healing, and embolization in vascular radiology of intracerebral AVMs, liver AVMs, angiodysplasia of colon, esophageal varices, sealing “pumping” gastrointestinal bleeders secondary to peptic ulcers, and the like. The subject invention is further useful for providing hemostasis in corneal transplants, nosebleeds, tonsillectomies, teeth extractions and other applications.

In each of the foregoing described applications, there is a break in the normal tissue integrity of the patient. The location of the break or the site of application of the FS is referred to herein collectively as a “wound site,” although it may not always be a wound per se. For example, an air leak is not necessarily a wound, nor is the addition of a prosthesis, but for the purpose of simplicity, they are collectively referred to herein as a “wound” because each occurs at a break in the normal tissue, and each is sealed or treated by application of the FS composition of the present invention.

In the preferred practice of the present invention, the FS composition is formulated and “instantly” converted, meaning concurrently with contact with the wound site, or within 300 seconds, preferably within 180-240 seconds, more preferably within 150-180 seconds, even more preferably within 100-150 seconds, and most preferably in less than 100 seconds the fibrin monomer forms and is converted to polymerized or partially polymerized fibrin, and/or noncross-linked fibrin is converted to cross-linked fibrin. In fact, the instant FS clot is typically formed in under 60 seconds, more often in under 30 seconds, and in the examples provided herein, the FS clot consistently formed in 8 to 30 seconds, most often in 9 to 12 seconds.

Thus, “instantly” or “concurrently” also refers to the fibrin-forming step occurring upon activation of the storage-stable fibrinogen component, within 300 seconds, preferably within 180-240 seconds, more preferably within 150-180 seconds, more preferably within 100-150 seconds, more preferably in less than 100 seconds, more preferably in less than 60 seconds, more preferably in less than 30 seconds, most preferably in 8 to 30 seconds, and typically in 9 to 12 seconds. However, even in the longest of times, the FS of the present invention is “instant” when compared to any prior art preparation because the fibrinogen is always ready-to-use in aqueous solution, as are the other component(s) without time consuming and difficult measuring or separate mixing and preparation of such component(s) from lyophilized or frozen formulations or from fresh blood or plasma samples.

In one preferred embodiment, the fibrin forming step and the contacting step at the wound site are “concurrent” meaning simultaneous, although polymerization may take some additional period to complete within the above-stated time ranges. However, the conversion step of the FS composition components to fibrin occurs within 60 seconds of activation and/or contact, preferably within 30 seconds, more preferably within 15 seconds, and yet more preferably within 10 seconds, most preferably within 0-10 seconds. Otherwise, the FS composition may flow away from the intended site.

Finally, instantly and concurrently can also mean that the conversion step commences prior to the contacting step, albeit not so far in advance of the contacting step that all of the fibrin monomer (resulting from activation of the storage-stable fibrinogen) has been polymerized or converted to cross-linked fibrin. For example, this embodiment of the invention could occur when the storage-stable fibrinogen component is activated by exposure to thrombin or a thrombin-like enzyme in the presence of calcium ions in a syringe-type device prior to application of the resulting combined composition to the wound site. If all of the resulting fibrin has been polymerized or converted to cross-linked fibrin prior to the contacting step, the composition no longer retains any fluidity and it can no longer form a satisfactory fibrin sealant, nor can it be any longer used for such purposes. Since it takes ideally takes less than about 30 seconds for the storage-stable fibrinogen component to be converted to the FS fibrin composition, the conversion step should not begin more than about 30 seconds, and preferably not more than about 3-10 seconds prior to the contacting step, unless a component such as a chaotropic agent has been added to delay the conversion. This embodiment is preferred because it ensures that the maximum amount of the FS composition will polymerize at the desired site, while at the same time form an excellent fibrin clot. As a result, the FS composition of the present invention that has been prepared from storage-stable fibrinogen eliminates many possible variables in the preparation of the sealant formulation, permitting instant application of the FS composition to the wound site under closely controlled conditions.

FS Delivery

The FS product of the present invention is conveniently formed by mixing at least two components just prior to use. The first component comprises storage-stable fibrinogen in ready-to-use aqueous solution, the second is an activator component, typically thrombin or a thrombin-like activator and calcium ions. Factor XIII and/or other additive components may also be included as described elsewhere in this specification. In general, the components are conveniently delivered using a two-syringe system, wherein the syringes are joined by a syringe-to-syringe connector having about a 1 mm or less diameter opening. Substantial uniformity can be achieved with simple, generally available equipment.

FS application in the prior art includes dual syringe devices, which mix the fibrinogen and thrombin as they exit from a single port, typically using a large needle to direct the flow onto the wound. However, such delivery systems are known to form clots within can cause needle and tube blockages. Known dual syringe systems are also awkward to fill and manipulate, and if there is inadequate mixing of the fibrinogen and thrombin components, the resulting clots may lack strength or elasticity. Because many wound sites leak significant amounts of fluid at the site, improperly formed fibrin seals may become ineffective or be flushed away. These problems are, however, overcome by the present invention because the stable fibrinogen is stored in aqueous solution, in ready-to-use form without mixing, measuring or time delay, and it may be directly stored in a pen-type syringe delivery device.

In the present invention, the components are mixed immediately prior to polymerization. The components may be formulated with concentrations that allow mixing the components in adequate volumes to simplify the final preparation of the adhesive, preferably the volumes are substantially equal. Conveniently, a dual-barrel syringe holder with a disposable mixing tip can be used. Alternatively, the two components can be mixed using two syringes as described above, or the components may be directly applied to the wound site, whereupon mixing occurs at the site. Preferably, however, the components are thoroughly mixed before delivery or as apart of the delivery process or form the FS composition at the time of delivery to the site.

The double-barrel syringe can be Y-shaped, thereby permitting the mixing of the FS composition from the storage-stable components, and the activation of the conversion step simultaneously with, or immediately prior to the contacting step. In the alternative, rather than a Y-shaped double-barrel syringe, a double-barrel syringe with two openings can be utilized. This permits the simultaneous contacting of the wound site and conversion to the FS polymer from the storage-stable components. In yet another alternative embodiment, the storage-stable components of the double-barrel syringe can be sprayed onto the desired site (see Kram et al., Amer. Surgeon, 57:381 (1991)). In yet another alternative embodiment, the storage-stable components are held in a single-barrel syringe separated by a non-porous material that is punctured, broken, dissolved or simply removed to allow the mixing of components just prior to delivery of the then-converted FS.

In the alternative, the fibrinogen is easily drawn into the delivery device from a larger container using standard drug delivery techniques used with medicaments delivered by syringe.

Fibrin Sealant Kits

Further provided in the present invention are kits for the ready-to-use delivery of an instant FS composition comprising at least two vials. One vial, which as previously noted is not glass, contains an aqueous solution of storage-stable fibrinogen at a concentration suitable for forming FS when mixed with an activator solution, such as thrombin or a thrombin-like composition, and a second vial contains an activator solution, which is preferably thrombin at a concentration suitable for forming FS when mixed with the contents of the storage-stable fibrinogen in the first vial. By “vial” is intended herein to include a barrel of a syringe and multiple vials include a multi-barrel syringe device.

The pH of the activator solution can be adjusted so that it will neutralize the fibrinogen component when the two are mixed, or a separate vial of neutralizing buffer may be provided to neutralize the fibrinogen component before it is mixed with the activator component.

A source of calcium ions, such as CaCl₂, is added to and stored with the contents of one of the at least two vials in an effective amount to ensure fibrin polymerization, preferably it is combined with the thrombin activator component. In the alternative, the calcium (CaCl₂) component is supplied in an additional vial. Additional components, such as a stabilizer and/or Factor XIII, and/or additives, such as a growth factor, drug, antibiotic, and the like are supplied by one or more additional vials, or alternately such additional components are added to and stored with the contents of the at least two vials.

In a preferred embodiment of the invention the storage-stable fibrinogen component is supplied at a concentration ranging from about 75-115 mg/ml, and thrombin is supplied at approximately 500 IU/ml. When present, CaCl₂ is supplied at approximately 40 mmol/liter. When present, a fibrinolysis inhibitor, such as aprotinin is supplied at approximately 3000 KIU/ml. Additional components, when present, are supplied at suitable concentrations as determined by the purpose for which they are added. For example, an antimicrobial component intended to stabilize the FS components per se are supplied at low concentrations as exemplified herein, whereas an antimicrobial composition intended for slow-release delivery to the patient to whom the FS is applied, would be supplied at a much higher concentration. Such amounts or concentrations can be determined or would be known to those skilled in the medical formulation art. Similarly, such an individual would know whether two or more specific components or additives can be combined and stored in a single vial without contra-indication, or whether the same components or additives will remain more independently active and storage-stable if supplied separately in individual vials.

The invention is further described by example. The examples, however, are provided for purposes of illustration to those skilled in the art, and are not intended to be limiting. Moreover, the examples are not to be construed as limiting the scope of the appended claims. Thus, the invention should in no way be construed as being limited to the following examples, but rather, should be construed to encompass any and all variations which become evident as a result of the teaching provided herein.

### EXAMPLES

### Example 1

### FS Clotting Assay for Preparations Using Fibrinogen Stored in Aqueous Solution at Room Temperature, Neutral pH

To evaluate the ability to rapidly prepare FS compositions from storage-stable, ready-to-use aqueous solutions of fibrinogen, the clotting activity of fibrin sealants prepared using fibrinogen that had been stored in aqueous solution for long periods of time were evaluated.

Bovine fibrinogen, bovine thrombin, buffer solutions, calcium chloride, sodium hydroxide and hydrochloric acid were purchased from Sigma Chemical Company, St. Louis. Mo. Human fibrinogen was certified to contain 53% protein (95% clottable) and 47% salts. Bovine fibrinogen was certified to contain 61% protein (97% clottable) and 39% salts.

Standard research grade fibrinogen contains salts used in the isolation and purification process. This includes sodium citrate and sodium chloride. Thus, a 40 mg/ml solution of fibrinogen contains, for example, 54 mM sodium citrate and 419 mM sodium chloride in addition to the fibrinogen. Additionally, sodium azide (0.025%) was added to each sample as an antimicrobial agent, although in a preferred embodiment of the present invention, no antimicrobial agent would be added to the fibrinogen, rather sterility would be preserved using known techniques.

The clotting assays were completed in the following manner in general accordance with Kasper, Proc. Symposium on Recent Advances in Hemophilia Care, Los Angeles. Calif. Apr. 13-15, 1989 (in Liss, N.Y., 1990). Aliquots (100 μl) of each fibrinogen sample were added to 4 ml polypropylene test tubes. Each sample was neutralized (pH 7.0-7.5) using 0.1 M sodium hydroxide, 0.2 M histidine buffer (pH 6.0) or 0.1 M hydrochloric acid (determined in preliminary studies using larger volumes). Thrombin was prepared as 200 units/ml with 1 M calcium chloride (3-6 mM excess of calcium over sodium citrate in fibrinogen preparations). The thrombin preparation was then diluted with 0.1 M histidine buffer (pH 7.2) to a final thrombin concentration of 100 units/ml (2.5 units of thrombin per mg of fibrinogen). All samples were assayed at room temperature (23±2° C.).

Clotting was measured by timing the reaction that occurred when 100 μl of thrombin was added to the fibrinogen sample (100 μl), and the mixture was vigorously mixed. Times were recorded when the solution turned to a viscous gel (a drastic slowing of the liquid being mixed) and to a solid clot (the point at which all liquid ceased movement upon mixing). The time to solid clot formation was often twice the time of gel formation.

In accordance with the described procedure, a manual clotting assay was performed at 25° C. by neutralizing the stored fibrinogen solutions; and adding thrombin (125 units/mg fibrinogen), and 3-5 mM excess CaCl₂ over citrate in the fibrinogen solution. The preparation was mixed vigorously, and the time required for a clot to form was measured as described above.

Clotting results using bovine fibrinogen in histidine buffer at pH 7.24, stored in aqueous solution at room temperature (˜23° C.) were as follows: using fibrinogen stored for 3 days a clot formed within 9 seconds, stored for 36 days a clot formed within 10 seconds, and stored for 72 days (more than 10 weeks) a clot formed within 9.5 seconds. Thus, the clotting assay results are consistent regarding the time needed to form a FS clot in a preparation prepared from fibrinogen stored at room temperature for long periods of time in ready-to-use aqueous solutions (at neutral pH).

### Example 2

### FS Clotting Assays Using Fibrinogen Solutions Stored at Two Temperatures, and a Range of pH Values

To evaluate the ability to rapidly prepare FS compositions from storage-stable, ready-to-use aqueous solutions of fibrinogen, the clotting activity was evaluated of fibrin sealants prepared using fibrinogen that had been stored in aqueous solution for long periods of time over a range of pH values (pH 6.50 to pH 9.87), at room temperature (˜23° C.) and refrigerated (4° C.). Duplicate solutions of fibrinogen were evaluated in clotting assays as described in the stability study in Example 1.

Clotting results are shown in Table 1 bovine fibrinogen (39 mg protein/ml) and in Table 2 for human fibrinogen (40 mg protein/ml), respectively prepared for storage in one of the following 0.1 M buffers: histidine, pH 6.0 or 7.2; Tris pH 8.16.

TABLE 1
Clotting times for bovine fibrinogen, stored at 23″ C. and 4″ C.

| Age in | Temp. in | Clotting Time (in seconds) | | | | |
|---|---|---|---|---|---|---|
| Days | ° C. | pH 6.5 | pH 7.36 | pH 8.2 | pH 9.04 | pH 9.87 |
| 4 | 23 | 12 | 13 | 15 | 12 | 210 |
| | 4 | 10 | 9 | 15 | 10 | Clotted |
| 7 | 23 | 10 | 10 | 11 | 11 | 240 |
| | 4 | 11 | 10 | 10 | 10 | Clotted |
| 22 | 23 | 9 | 10 | 10 | >300 | >300 |
| | 4 | Partial clot | Partial clot | Clotted | Clotted | Clotted |
| 97 | 23 | 10 | 100 | >300 | >300 | Clotted |
| | 4 | Clotted | Clotted | Clotted | Clotted | Clotted |

NT = not tested.
“Clotted” refers to spontaneous clotting, absent addition of thrombin.

TABLE 2
Clotting times for human fibrinogen stored at 23° C. and 4° C..

| Age in | Temp. in | Clotting Time (in seconds) | | | | |
|---|---|---|---|---|---|---|
| Days | ° C. | pH 6.32 | pH 7.13 | pH 8.04 | pH 8.79 | pH 9.43 |
| 4 | 23 | 10 | 10 | 11 | 12 | 120 |
| | 4 | 10 | 10 | 9 | 10 | Clotted |
| 7 | 23 | 10 | 10 | 9 | 11 | 240 |
| | 4 | 10 | 9 | 8 | 9 | 12 |
| 22 | 23 | 10 | 8 | 10 | >300 | >300 |
| | 4 | 10 | 8 | 9 | NT | NT |
| 97 | 23 | 30 | >300 | >300 | >300 | >300 |
| | 4 | 18 | 10 | 10 | 11 | >300 |
| 149 | 23 | NT | >300 | >300 | NT | NT |
| | 4 | 15 | 135 | 30 | >300 | >300 |

NT = not tested.
“Clotted” refers to spontaneous clotting, absent addition of thrombin.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

1. A ready-to-use, instantly available fibrin sealant (FS) composition prepared from a storage stable, aqueous fibrinogen solution component and an activated thrombin or thrombin-like component.

2-18. (canceled)
